(19) United States
(12) Patent Application Publication
(10) Pub. No.: US 2011/0033346 A1
(43) Pub. Date: Feb. 10, 2011

(54) AIR CLEANER WITH PHOTO-CATALYTIC OXIDIZER

(76) Inventors: Johns R. Bohlen, Rickman, TN (US); Wang Bozoo, Dongguang (CN)

Correspondence Address: Winston & Strawn LLP;Patent Department, 1700 K Street, N.W., Washington, DC, 20006, US

(21) Appl. No.: 12/535,520

(22) Filed: Aug. 4, 2009

(51) Int. Cl. A61L 9/18; A61L 9/20; B01J 19/00

(52) U.S. Cl. 422/186.3; 422/311

(57) ABSTRACT

An air cleaner comprising an air channel in the air cleaner; an air moving unit configured to create an airflow in the air channel; a photo-catalytic oxidation (PCO) element disposed in the airflow; and a Ultraviolet A (UV-A) Light Emitting Diode (LED) to illuminate the PCO element is described. The PCO element can be a substrate comprising: a first coating comprising a VOC decomposing catalyst; a second coating comprising an ozone decomposing catalyst; and a third coating comprising titanium dioxide.

### TECHNICAL FIELD

The present invention relates to an air cleaner, and more particularly, to an air cleaner with a photo-catalytic oxidizer adapted to clean a filtering media.

### BACKGROUND

Air cleaners and purifiers are widely used for removing foreign substances from the air. The foreign substances can include pollen, dander, smoke, pollutants, dust, etc. In addition, an air cleaner can be used to circulate room air. An air cleaner can be used in many settings, including at home, in offices, etc.

One prior art air cleaner includes a traditional pre-filter, an electrostatic precipitator, a fan unit, and a post-filter. The pre-filter, the electrostatic precipitator, the fan unit, and the post-filter are independent devices, wherein each component can be independently installed and removed. Airflow through the tower prior art air cleaner first encounters the pre-filter, then the electrostatic precipitator, the fan unit, and finally the post-filter. The pre-filter removes larger objects from the airflow, such as debris that could block or clog the electrostatic precipitator. The pre-filter is typically rather coarse, and is designed to minimally impede the incoming airflow. The electrostatic precipitator removes dirt and debris by electrostatic attraction to electrode plates, as previously discussed. The post-filter is typically a fine filter that is designed to remove any small particulate remaining in the airflow.

This prior art air cleaner has drawbacks. The post-filter is located after the fan unit. The post-filter is included to remove any dirt and debris that is not removed by the pre-filter and by the electrostatic precipitator. Some dirt and debris is still in the airflow when it reaches the post-filter. The post-filter utilizes catalysts to remove volatile organic compounds (VOCs), ozone and/or odors from the airflow. However, over time the catalyst surface gets covered with particulates. This results in reduced cleaning efficiency and reduced life span of the post-filter. There exists a need to increase the lifespan of a post-filter in an air cleaner.

The prior art also uses ultraviolet light (UV) light bulbs which have a shorter life span than the air cleaner. As such, they have to be replaced when they burn out adding costs and inefficiencies to the air cleaner. UV light bulbs also have a large footprint. UV light bulbs can also require a large volume within an air cleaner. As such, UV light bulbs can constrain the design of the air cleaner. UV Light Emitting Diodes (LEDs) have a greater life span than conventional UV light bulbs. Some UV LEDs can last the lifetime of the air cleaner. There exists a need to provide for longer lasting UV light sources in air cleaners.

### SUMMARY

According to one embodiment, an air cleaner can comprise an air channel in the air cleaner; an air moving unit configured to create an airflow in the air channel; a photo-catalytic oxidation (PCO) element disposed in the airflow; and a Ultraviolet A (UV-A) LED to illuminate the PCO element. The PCO element can cause decomposition of water vapor in the airflow. The PCO element can be supported by pellets, mesh, screen, cloth, paper, a porous substrate or a combination thereof. The PCO element can include titanium dioxide. In some embodiments, the UV-A LED can be disposed outside of the air channel. In some embodiments, the air cleaner can include a decomposing element disposed in the air flow. In some embodiments, the air cleaner can include a filter; and a decomposing element, wherein the PCO element and the decomposing element are supported by the filter. The filter can include first and second opposite faces. The decomposing element can be disposed on the first face of the filter and the PCO element can be disposed on the second face of the substrate. In some embodiments, the filter can include air passages between the first face and the second face. The honeycomb can have a length that is greater than a length of the PCO coating on the honeycomb. The decomposing element can be disposed in the airflow downstream of the PCO element. The decomposing element can be an ozone decomposition element. The decomposing element can be a VOC decomposition element. The decomposing element can be an odor removal element. The decomposing element can be an ozone decomposition element and a VOC removal element. The UV-A LED can be operable to emit UV light comprising wavelengths from 320 nanometers (nm) to 400 nm. The UV-A light emitting diode can emit UV light having an intensity sufficient to energize the titanium dioxide to cause photo-catalytic oxidation. In some embodiments, the air cleaner can include an air filter element adapted to capture particulates having an average diameter less than 1 millimeter (mm) from the airflow. In some embodiments, the air cleaner can include an electrostatic precipitator disposed in the air flow upstream of the PCO element.

According to one embodiment, an air cleaner can comprise: an air channel in the air cleaner; an air moving unit configured to create an airflow in the air channel; an VOC decomposing element located in the air channel; a PCO element comprising titanium dioxide in contact with the VOC decomposing element; and a UV LED to illuminate the PCO element. When illuminated by the UV LED a reaction in the PCO element cleans the VOC decomposing element. The UV LED can have a mean time between failure (MTBF) of greater than one year or 84600 hours. The VOC decomposing element can have a MTBF of greater than one year or 84600 hours. The PCO element can have a MTBF of greater than one year or 84600 hours. The PCO element can span the air channel.

According to one embodiment, a substrate for being disposed in an air cleaner can comprise: a first coating comprising a VOC decomposing catalyst; a second coating comprising an ozone decomposing catalyst; and a third coating comprising titanium dioxide. The substrate can include two opposite faces and a plurality of air channels extending between the two opposite faces. A length of one of the air channels can be greater than a length of the third coating. In some embodiments, the substrate can include a frame disposed around the substrate. The substrate can be formed using a plurality of serpentine metal sheets joined together with a plurality of gaps between adjacent metal sheets.

### BRIEF DESCRIPTION OF THE DRAWINGS

The same reference number represents the same element on all drawings. It should be noted that the drawings are not necessarily to scale. The foregoing and other objects, aspects, and advantages are better understood from the following detailed description of a preferred embodiment of the invention with reference to the drawings, in which:

FIG. 1A illustrates a convertible air cleaner according to an embodiment in a tower orientation;

FIG. 1B illustrates a bottom view of a convertible air cleaner according to an embodiment;

FIG. 1C illustrates a convertible air cleaner according to an embodiment in a non-tower orientation;

FIG. 2 illustrates an exploded view of a convertible air cleaner according to an embodiment;

FIG. 2A illustrates a view of a control panel according to an embodiment;

FIG. 2B illustrates a view of a remote control according to an embodiment;

FIG. 3 illustrates a cross-sectional view of a convertible air cleaner according to an embodiment;

FIG. 4 illustrates a prospective view of a portion of an air filter according to an embodiment;

FIG. 5A illustrates an embodiment of a high density UV LED board;

FIG. 5B illustrates an embodiment of a low density UV LED board;

FIG. 6 illustrates a cross-sectional block diagram of a convertible air cleaner according to an embodiment;

FIG. 7 illustrates an embodiment of a filter;

FIG. 7A illustrates an embodiment of a filter surface; and

FIG. 7B illustrates an embodiment of a filter surface including a PCO element.

### DETAILED DESCRIPTION

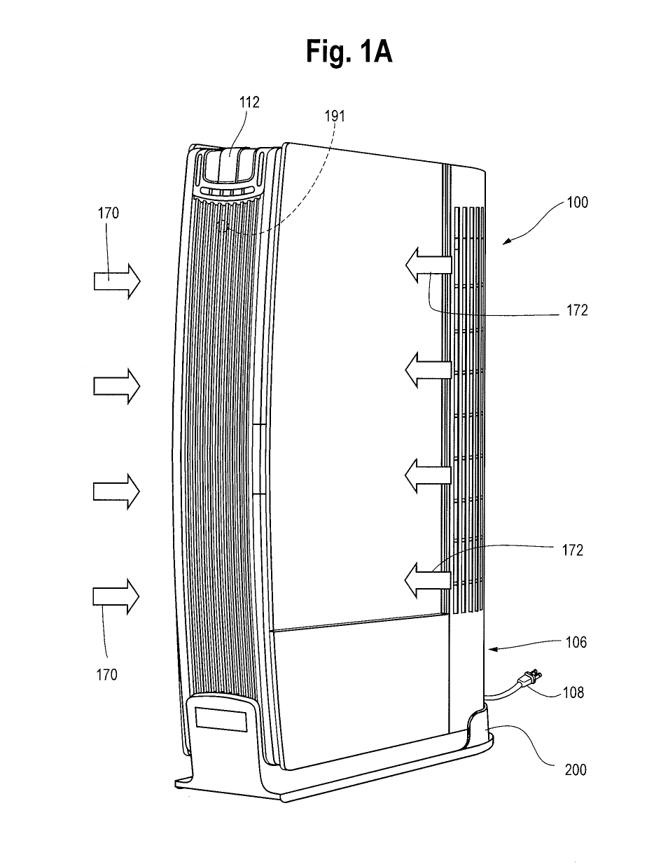

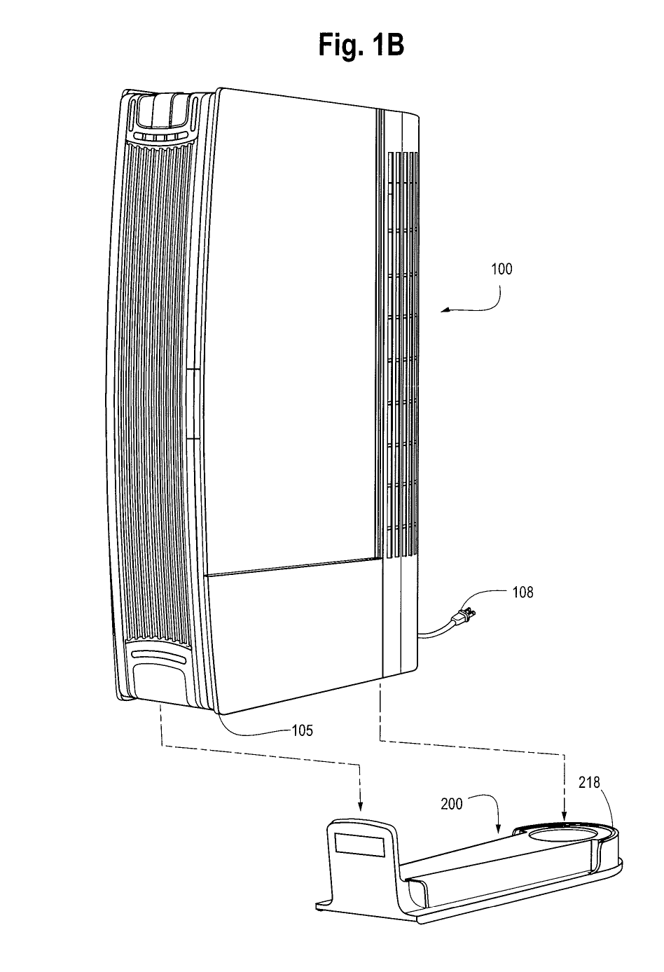

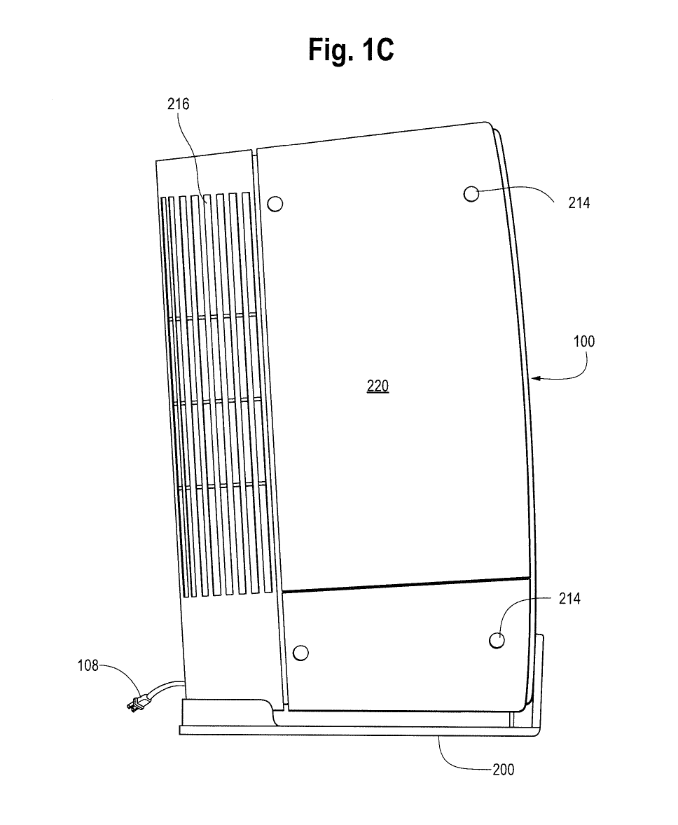

FIGS. 1-7 and the following descriptions depict specific embodiments to teach those skilled in the art how to make and use the best mode of the teachings. For the purpose of teaching these principles, some conventional aspects have been simplified or omitted. Those skilled in the art will appreciate variations from these embodiments that fall within the scope of the teachings. Those skilled in the art will also appreciate that the features described below can be combined in various ways to form multiple variations. As a result, the teachings are not limited to the specific embodiments described below, but only by the claims and their equivalents.

As used herein, the term “filter” refers to the extraction or removal of impurities or particulates from the air. The impurities or particulates can include, but are not limited to dust, dirt, debris, volatile organic compounds, ozone, carbon dioxide, radon, carbon monoxide, pollen, spores, microbes, viruses, etc. The impurities or particulates can be macroscopic or microscopic.

FIG. 1A shows an air cleaner 100 according to an embodiment. Air cleaner 100 includes a housing 106 and optionally a stand 200. Housing 106 can include an air inlet 110, a housing door 102, a control panel 112, a remote sensor 191 and an outlet 160 disposed therein or thereupon. An air inflow 170 is drawn in through inlet 110. The air is cleaned inside air cleaner 100, and the cleaned air is exhausted from air outlet 160. Additionally, a power cord 108 can extend from housing 106.

In some embodiments, air cleaner 100 can be generally vertically positioned when housing 106 is positioned in removable stand 200 converting air cleaner 100 into a tower air cleaner. In some embodiments, air cleaner 100 can be generally horizontally positioned when housing 106 is not positioned in removable stand 200. In various embodiments, air cleaner 100 can be substantially cylindrical, substantially elliptical, substantially cuboidal, or substantially rectangular-cuboidal, or combinations thereof, in shape. The exterior or outer face of housing 106 can be planar, circular, curvilinear, arcuate, or combinations thereof in shape. Air inlet 110 can be planar, circular, curvilinear, arcuate, or combinations thereof in shape. Air outlet 160 can be planar, circular, curvilinear, arcuate, or combinations thereof in shape. In one embodiment, air inlet 110 can be curvilinear and air outlet 160 can be arcuate in shape.

FIG. 1B illustrates how air cleaner 100 can be coupled to stand 200. Stand 200 can provide a slot 218. Slot 218 can complement lip 105 of housing 106. Slot 218 can receive lip 105 of housing 106 when air cleaner 100 is deployed in a tower orientation. FIG. 1C illustrates air cleaner 100 according to another embodiment. A bottom exterior or outer face 222 of housing 106 can support non-skid surface 214 to aid in stabilization of air cleaner 100 when in the non-tower position. Alternatively, non-skid surface 214 can comprise a portion of outer face 222 of housing 106. Non-skid surface 214 can have a higher coefficient of friction compared to the rest of housing 106. Non-skid surface 214 can be formed from texturing the outer face of housing 106, such as an applied enamel or paint, or can an external application of rubber, plastic, or other suitable material which can be applied. Non-skid surface 214 can be applied as strips, stripes, cross-hatches, or circular foot pads. Non-skid surface 214 can be permanent or removable. Along the bottom outer face 222, grille 216 can be a false grille and have no opening for airflow therein. As such, grille 216 can provide a pleasing appearance without allowing airflow to be directed to a surface that is supporting air clearing 100.

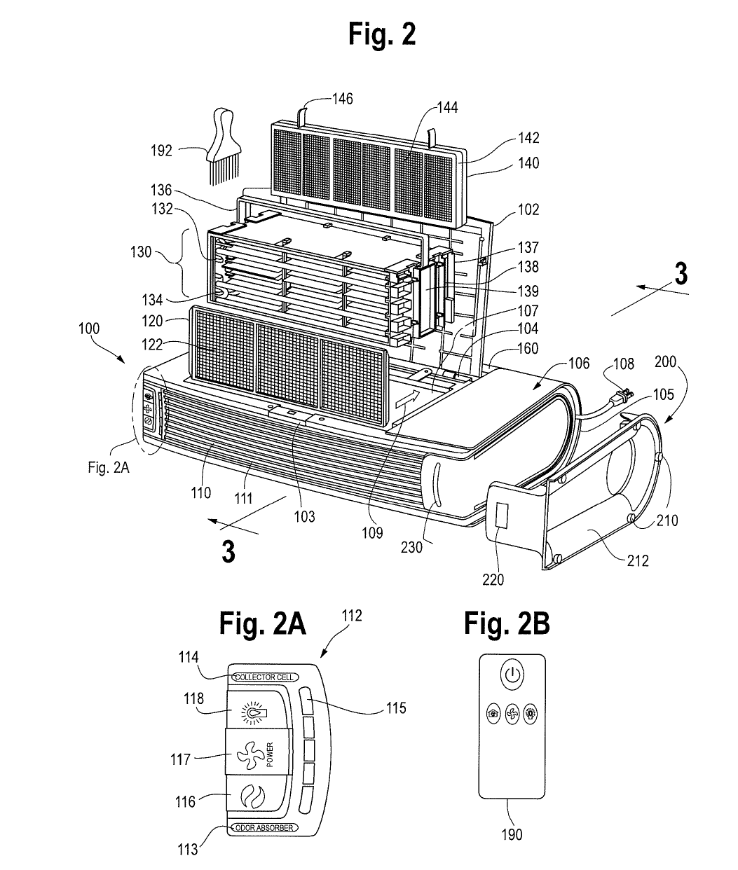

FIG. 2 shows an exploded view of air cleaner 100. Housing 106 can define an air channel 107 extending from air inlet 110 to air outlet 160. Air channel 107 can extend substantially linearly between air inlet 110 and air outlet 160. Obstructions or obtrusions into air channel 107 are minimized. In some embodiments, air cleaner 100 can include a door 102 that attaches to housing 106 via hinges 104. Door 102 can be latched. Door 102 can be opened by, for example, pushing button 103 to disengage the latch. In some embodiments, a lip 105 can be disposed along a face of housing 106. Lip 105 can be inserted into stand 200. In an embodiment, air inlet 110 is substantially opposite of air outlet 160. Air inflow 170 enters air cleaner 100 through air inlet 110. A cleaning brush 192 can be provided to clean inlet grille 111 or outlet grille 161.

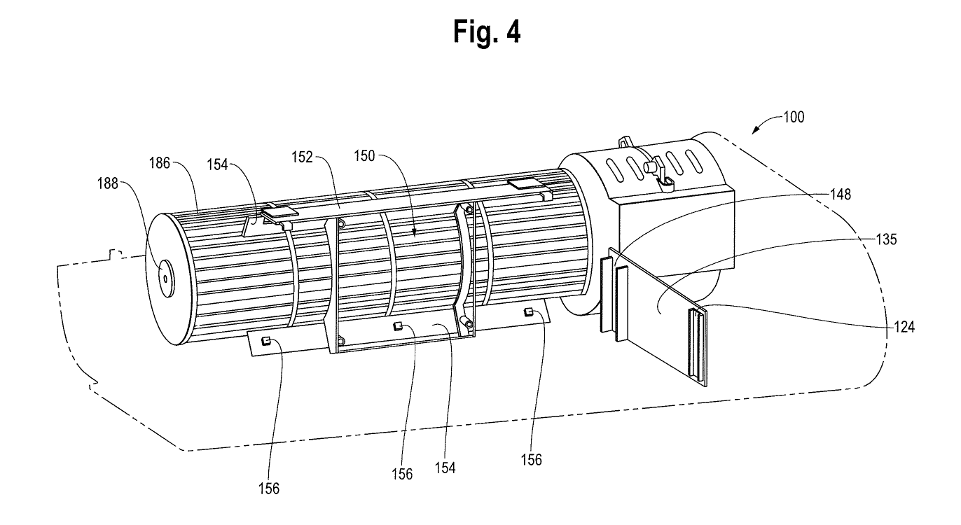

In some embodiments, air cleaner 100 can include a pre-filter 120, an electrostatic precipitator 130, a post-filter 140, and a fan unit 180 (shown in FIG. 4) all disposed in air channel 107. Air inflow 170 is termed an airflow 109 within air clearer 100. In an embodiment, airflow 109 encounters electrostatic precipitator 130 after encountering pre-filter 120. In an embodiment, airflow 109 encounters post-filter 140 after encountering electrostatic precipitator 130. In some embodiments, airflow 109 encounters an UV Light Emitting Diode (LED) assembly 150 (shown in FIG. 4) after encountering post-filter 140. In some embodiments, airflow 109 does not encounter UV LED assembly 150. In some embodiments, airflow 109 then encounters fan unit 180. When airflow 109 leaves air cleaner 100, it is termed air outflow 172.

Pre-filter 120, electrostatic precipitator 130, post-filter 140, UV LED assembly 150 and fan unit 180 can be independent units. Pre-filter 120, electrostatic precipitator 130, post-filter 140, UV LED assembly 150 and fan unit 180 can comprise units that are removably or permanently mounted in air channel 107. Pre-filter 120, electrostatic precipitator 130, post-filter 140, UV LED assembly 150 and fan unit 180 can comprise non-limiting combinations of removable and non-removable units that are mounted in air channel 107. Due to the independent nature of pre-filter 120, electrostatic precipitator 130, post-filter 140, and UV LED 150 assembly, each component can be independently installed and can be independently removed. In addition, air cleaner 100 can be assembled into various configurations by selection of the various cleaning components for a particular application.

Pre-filter 120, electrostatic precipitator 130, post-filter 140, UV LED assembly 150, and fan unit 180 can be received in an air cleaner chassis or frame by some manner of receptacle, slot(s), rail(s), etc., and can be easily and quickly inserted and removed. In one embodiment, pre-filter is received in a pre-filter receptacle 124, shown in FIG. 4, in air channel 107. In one embodiment, electrostatic precipitator 130 is received in an electrostatic precipitator receptacle 135, shown in FIG. 4, in air channel 107. In one embodiment, post-filter 140 is received in a post-filter receptacle 148, shown in FIG. 4, in air channel 107. In one embodiment, UV LED assembly 150 is received in a UV light receptacle 158, shown in FIG. 4, in air channel 107. One or more of the various receptacles can comprise drop-in receptacles. One or more of the various receptacles can comprise slide-in receptacles. One or more of the various receptacles can comprise receptacles that fixedly receive a component. It should be understood that other receptacle configurations are contemplated and are within the scope of the description and claims. The various receptacles can hold their respective units permanently or removably.

Pre-filter 120, electrostatic precipitator 130, post-filter 140, UV LED assembly 150, and fan unit 180 can be held in a substantially vertical position or in a substantially horizontal position. For example, pre-filter 120, electrostatic precipitator 130, post-filter 140, UV LED assembly 150, and fan unit 180 car. be retained vertically when air cleaner 100 is in a tower position. Further, pre-filter 120, electrostatic precipitator 130, post-filter 140, UV LED assembly 150, and fan unit 180 can be retained horizontally when air cleaner 100 is in a non-tower position. The various receptacles can ensure that the held component does not fall out of the receptacle when air cleaner 100 is in the tower or non-tower orientation.

Pre-filter 120 can comprise a pre-filter element 122. In one embodiment, pre-filter 122 comprises a fiber, a mesh, cloth, paper, a woven filter, or a combination thereof. Pre-filter 120 can comprise a High Efficiency Particulate Air (HEPA) filter (typically able to remove 99.7% of particulates to about 0.3 micron in diameter), an allergen air filter, an electrostatic air filter, a charcoal filter, an anti-microbial filter, or other filtering media known in the art. In addition, pre-filter 120 can be treated with a germicide, fungicide, bactericide, insecticide, etc., in order to kill germs, mold, bacteria, viruses, and other airborne living organisms (including microorganisms). Pre-filter 120 can have length L, height H, and width W. Pre-filter 120 can be capable of filtering impurities or particulates with an average diameter of at least 0.1, 0.3, 0.5, 1.0, 5.0, 10.0, 100 microns or greater, including impurities or particulates with an average diameter of 0.001, 0.01, 0.1, 1.0 millimeters or greater.

Electrostatic precipitator 130 removes dirt and debris from the airflow by electrostatic attraction. Electrostatic precipitator 130 can be removed and cleaned. An electrostatic precipitator operates by creating a high voltage electrical field. Dirt and debris in the air becomes ionized when it is brought into the electrical field by an airflow. Charged electrodes in the electrostatic precipitator air cleaner, such as positive and negative plates or positive and grounded plates, attract the ionized dirt and debris. Because the electrostatic precipitator comprises electrodes or plates through which airflow can easily and quickly pass, only a low amount of energy is required to generate the airflow. As a result, foreign objects in the air can be efficiently and effectively removed. Electrostatic precipitator can comprise corona wires or corona plates for ionizing the air particles. Electrostatic precipitator 130 can have length L, height H, and width W. Electrostatic precipitator 130 can be capable of filtering impurities or particulates with an average diameter of at least 0.1, 0.3, 0.5, 1.0, 5.0, 10.0, 100 microns or greater, including impurities or particulates with an average diameter of 0.001, 0.01, 0.1, 1.0 millimeters or greater.

Electrostatic precipitator 130 can further comprise one or more handles or knobs 136. Handle 136 can be used to easily grasp electrostatic precipitator 130 for installation and removal from electrostatic precipitator receptacle for cleaning or replacement. Electrostatic precipitator 130 is capable of generating ozone by ionization of oxygen. The ionization transforms stable (O₂) molecules in the air into ozone molecules (O₃). Subsequently, the third oxygen atom of the ozone molecules enter into destructive reactions with contaminants in the vicinity by oxidizing compounds they come into contact with. The oxidation can add oxygen molecules to these contacted compounds during the oxidation reaction. Ozone is a powerful oxidizer because it is not a stable molecule. Ozone molecules spontaneously return to a stable, molecular state by releasing their third oxygen atoms. However, the spontaneous breakdown of ozone does not occur immediately, and substantial amounts of ozone can linger in the airstreams for some time. One of the great advantages of ozone is that it is not selective in the reactions it initiates. Ozone neutralizes harmful volatile organic compounds (VOCs) by oxidizing them. Ozone also destroys pathogens (microorganisms), either by reducing or destroying them or by cell lysing or oxidation. Another beneficial effect of ozone is that ozone treatment of the air can remove some troublesome odors.

The ozone generated in air cleaner 100 can comprise any source of ozone. For example, air cleaner 100 in one embodiment comprises an air ionizer (not shown) that is designed to generate significant levels of ozone molecules in order to kill living material in the air and to decompose unwanted or unhealthy material in the air. It is desirable to remove this ozone from airflow 109 of air cleaner 100. In one embodiment, an ozone generating device comprises electrostatic precipitator 130. Electrostatic precipitator 130 can produce relatively small amounts of ozone as a by-product. In yet another embodiment, air cleaner 100 comprises a UV light source that generates ozone by illuminating a catalyst.

Electrostatic precipitator 130 includes a frame 137, and charge and collection plates 134. The frame 137 in one embodiment includes support projections 138 that slide into and are received by the support projection channels (not shown) of air cleaner 100. Support projections 138 can be used by air cleaner 100 in order to hold, retain, and steady electrostatic precipitator 130. Such a design allows a gap between the top of the cell and the inlet to fan unit 180 to be minimized whether air cleaner 100 is in a tower or a non-tower orientation. This advantageously allows less by-pass of airflow 109 around post-filter 140 and achieves better cleaning of the airflow. Support projections 138 can comprise projections formed on the frame (not shown). In one embodiment, support projections 138 can be formed substantially at a top region of the frame (not shown). In another embodiment, support projections 138 can be formed traversing the entire length of the frame (not shown). However, it should be understood that support projections 138 can be located anywhere on frame 137. Support projections 138 can comprise projections that have an outward dimension D and a length L.

Electrostatic precipitator 130 can also include a handle 136. Handle 136 enables electrostatic precipitator 130 to be gripped for insertion and removal in air cleaner 100. Handle 136 can be permanent or removable. If handle 136 is removable, it may be stored within housing 106. In one embodiment, handle 136 can slide along a track 139 on electrostatic precipitator 130, which enables the handle to be raised or lowered thereby reducing the total area of electrostatic precipitator 130 when inserted into air cleaner 100. Handle 136 can also be hinged, allowing the handle to lay flat on top of electrostatic precipitator 130 thereby reducing the total area of electrostatic precipitator 130 when inserted into air cleaner 100. Handle 136 can be made from the same material as electrostatic precipitator, or can be made from non-conductive materials.

Post-filter 140 can comprise a post-filter frame 142 adapted to support a post-filter substrate 144. In some embodiments, post-filter 140 can comprise metal. Post-filter 140 can include a grounding tab 146, so any static electrical charges build in for example post-filter substrate 144 can be discharged. In some embodiments, grounding tab 146 can be used as a handle to ease removal post-filter from air cleaner 100.

As seen in FIGS. 1A-1C and FIG. 2, air inlet 110 can comprise a substantially rectangular inlet, wherein air inflow 170 travels substantially linearly into air inlet 110, through grille 111 and through pre-filter 120. Cleaned air outflow 172 can travel substantially linearly outward from air outlet 160 through grille 161. Cleaned air outflow 172 can travel substantially horizontally, vertically or can be exhausted at an angle from horizontal, depending on whether air cleaner 100 is in a tower or a non-tower position. Cleaned air outflow 172 can exit substantially opposite the direction of air inflow 170. Grille 111 or 161 can include louvers, slats, bars, mesh, or wire. The louvers, slats, bars, mesh, or wire of grille 110 or 161 can be permanent, or replaceable or combinations thereof. The louvers, slats, bars, mesh, or wire and can be fixed or stationary, or combinations thereof, and are capable of directing the direction of airflow into air channel 107 through air inlet 110, and out of air outlet 160. The direction of airflow out of air outlet 160 can be 180, 160, 140, 120, 90, 60, 45, 30 or less degrees away from air cleaner 100. Air inlet 110 is shown as being centered on housing 106. However, it should be understood that alternatively the relative position of air inlet is movable within housing 106, and can alternately be moved to other housing portions.

A control panel 112 may be located on the outer face with the air inlet 110. Control panel 112 optionally includes buttons, switches, dials, and indicator lights and the like. Control panel 112 may optionally include buttons for an air ionizer 116, fan unit 117, and night light 118, for example. In some embodiments, button 112 can be used to control UV LED assembly 150. Control panel 112 may further optionally include indicator lights 113 and 114 which alert the user to clean pre-filter 120, electrostatic precipitator 130, post-filter 140, or to change a UV LED assembly 150. Control panel 112 can include indicator light 115 to display a fan speed. Control panel 112 can be advantageously disposed on housing 106, thus allowing a user to easily reach the buttons in a tower or a non-tower orientation without resorting to contorting or moving air cleaner 100 to access buttons 116, 117, and 118. Control panel 112 can be advantageously disposed on housing 106, thus allowing a user to easily view indicators 113, 114, and 115 in a tower or a non-tower orientation without resorting to contorting or moving the air cleaner. Air cleaner 100 can be provided with a remote sensor 109 (shown in FIG. 2) and a remote control 190 (shown in FIG. 2B) to remotely control air cleaner 100. Air cleaner 100 can be configured to receive power from an external power source or battery. The external power source can generate a direct current (DC) high voltage for electrostatic precipitator 130. The voltage is typically on the order of thousands of volts or even tens of thousands of volts.

Air cleaner 100 can comprise a control circuit (not shown) that can control the overall operation of air cleaner 100. The control circuit can be connected to control panel 112 as shown in FIG. 2A. In some embodiments, the control circuit can accept user input from remote control 190 via remote sensor 109. The control circuit can receive user inputs through control panel 112. The control circuit can generate outputs to the control panel 112, such as lighting indicator lights, for example. In addition, in some embodiments the control circuit is connected to fan unit 180, the high voltage power supply (not shown), UV light bulb assembly 150, housing door 102 and a shut-down circuit (not shown). The control circuit in some embodiments can sense a state of one or more of these components. The control circuit can in some embodiments send signals, commands, or the like to one or more these components. The control circuit in some embodiments can receive signals, feedback, or other data from these components. The control circuit in some embodiments is coupled to and communicates with the shut-down circuit. The control circuit can shutdown power to fan assembly 180, electrostatic precipitator 130, and/or the high-power power supply when door 102 is opened. In some embodiments, the control circuit can shutdown power to fan assembly 180, electrostatic precipitator 130, and/or the high-power power supply when one of the filtering components needs cleaning or servicing.

The shut-down circuit can be configured to monitor an electrical current supplied to electrostatic precipitator 130, remove electrical power to electrostatic precipitator 130 if the electrical current exceeds a predetermined cell current threshold for a predetermined time period, and generate an indication, such as due to arcing. The shut-down circuit can be located between the high voltage power supply and electrostatic precipitator 130, wherein the shut-down circuit can interrupt the electrical power that is supplied to electrostatic precipitator 130. As a result, the shut-down circuit can make or break the power lines between the high voltage power supply and electrostatic precipitator 130. It should be noted that electrical power to fan unit 180 can be maintained or can be terminated when the electrical power to electrostatic precipitator 130 is removed. The control circuit can illuminate a clean electrostatic precipitator 114 indicator based on a run time of electrostatic precipitator 130. In some embodiments, air cleaner 100 can be operated without electrostatic precipitator 130 disposed therein. When air cleaner 100 operates without electrostatic precipitator 130, the control circuit can be programmed to not increment the run-time of electrostatic precipitator 130.

After an arc or short has exceeded the predetermined time period, an indication can be generated. The indication in one embodiment comprises a light that is illuminated. The indication can include a steady illumination or a blinking illumination. Alternatively, other trouble indications can be generated, including audible signals. The indication can be generated until a power cycle of air cleaner 100 occurs

The shut-down circuit can be configured to monitor the open or closed status of housing door 102 and remove electrical power to UV LED assembly 150 if housing door 102 is ajar when the power is on. Alternately, the shut-down circuit can be configured to monitor the open or closed status of housing door 102 and remove electrical power to fan unit 180 if housing door 102 is ajar when the power is on. It should be noted that electrical power to fan unit 180 can be maintained or can be terminated when the electrical power to the UV LED assembly 150 is removed. Alternatively, it should also be noted that electrical power to the UV LED assembly 150 can be maintained or can be terminated when the electrical power to fan unit 180 is removed. The shut-down circuit can be configured to monitor the open or closed status of housing door 102 and remove electrical power to the UV LED assembly 150 and fan unit 180 if housing door 102 is ajar when the power is on.

Power can be restored to the circuit when a power cycle occurs. The power cycle can comprise a person pressing the power button. In addition or alternatively, the power cycle can comprise a person unplugging air cleaner 100 from a power outlet. Other power cycle actions are contemplated and are within the scope of the description and claims.

Once a power cycle has occurred, electrical power is restored to the component that had been interrupted. Thus, power is restored to electrostatic precipitator 130, fan unit 180, UV light bulb assembly 150, etc., and the specific component therefore resumes operation. In addition, the indication is terminated.

Additionally, FIG. 2 also illustrates a bottom face 212 of removable stand 200 that can include a non-skid surface 210 to aid in stabilization of air cleaner 100 in the tower position. Non-skid surface 210 can comprise the entire outer face or housing 106, or can comprise discrete portions. Non-skid surface 210 can have a higher coefficient of friction compared to the rest of stand 200. Non-skid surface 210 can be formed from texturing the outer face of stand 200, such as an applied enamel or paint, or can an external application of rubber, plastic, or other suitable material which can be applied. Non-skid surface 210 can be applied as strips, stripes, cross-hatches, or circular foot pads. Non-skid surface 210 can be permanent or removable. In some embodiments, lip 105 of air cleaner 100 can be formed using a non-skip surface and air-cleaner 100 can be placed in a tower orientation without a stand. In other embodiments, a non-skip surface can be applied to lip 105. Stand 200 can comprise a transparent portion 220. A night light 230 disposed on housing 106 can be visible through transparent portion 220.

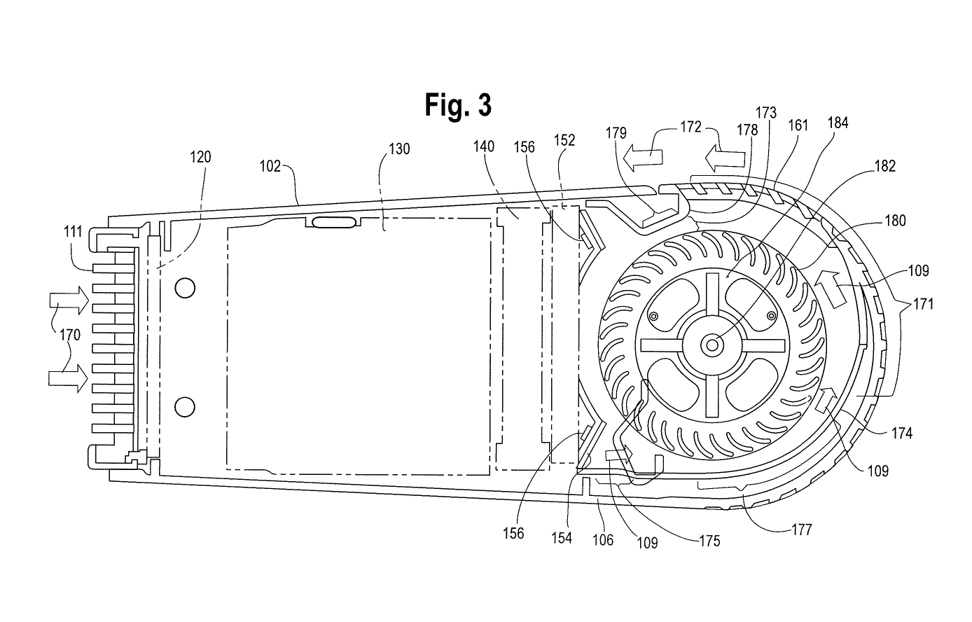

FIG. 3 shows a cross-section of air cleaner unit 100 according to an embodiment 100. In the embodiment shown, air cleaner 100 is the tower air cleaner 100 of FIG. 2. Other air cleaner configurations are contemplated and are within the scope of the description and claims. It should be understood that air channel 107 (and the components of air channel 107 as previously discussed) can be used in other air cleaner arrangements. Air cleaner 100 in this embodiment can include housing 106, wall 174, and door 102. In this embodiment, fan unit 180 draws air into the unit and the air flows through front grille 111 of air inlet 110, through pre-filter 120, through electrostatic precipitator 130, through post-filter 140, and clean air exits via air-outlet 160 and is directed into the surrounding environment via air outlet grille 161. Door or doors 102, when installed, can operate to retain the electrostatic precipitator 130 in the electrostatic precipitator receptacle (not shown) of air cleaner 100.

Air channel 107 includes a shaped input region 175. Shaped input region 175 is located downstream of the air cleaning components and upstream of the fan unit 180. Airflow 109 entering the shaped input region 175 is substantially linear. Airflow 109 in one embodiment is substantially constrained in a linear manner by the parallel plate construction of electrostatic precipitator 130. As a result, airflow 109 travels through shaped input region 175 and meets fan unit 180 in a substantially radial manner (see arrows). Shaped input region 175 in some embodiments is somewhat tapered and as a result airflow 109 will increase in velocity before airflow 109 meets fan unit 180.

Air channel 107 further includes a curved transition region 177. Curved transition region 177 is downstream of shaped input region 176. The fan unit 180 resides in the curved transition region 177. The fan unit 180 accelerates the air entering from shaped input region 176. Curved transition region 177 transitions airflow 177 from an air inlet orientation to air outlet orientation.

Air channel 107 further includes an expansion output region 171. Expansion output region 171 is downstream of curved transition region 177 (although the two regions include some overlap). Curved transition region 177 can expand in cross-sectional area (and therefore in volume), while output region 171 can be at least partially curved. Output region 124 in some embodiments comprises a partial spiral or scroll shape of expanding or increasing radius. Output region 171 comprises a larger cross-sectional area adjacent to fan unit 180 that allows the airflow to expand and slow down. Output region 171 directs airflow 109 so as to control the rate of expansion and minimize counter-flow and turbulence. As a consequence, noise generated by air cleaner 100 is significantly reduced. For example, a reduced airspeed at air outlet 161 reduces turbulence and noise at outlet grille 161. Airflow 109 leaving fan unit 180 travels through output region 171 to the outlet grille 161.

Air channel further includes a cutoff region 179. The cutoff region 179 separates shaped input region 175 from output region 171. Housing 106 in cutoff region 179 is formed adjacent to the impeller 180, separated by a predetermined cutoff gap 173. Cutoff gap 173 is relatively small. In a preferred embodiment cutoff gap 173 is about 5 mm, about 3 mm, about 2 mm, or smaller. As a result, air pressure in cutoff gap 173 is relatively high. The high pressure in cutoff gap 131, in combination with a rounded nose 178 (see below), serves to separate airflow 109 from fan unit 180. However, the shape of cutoff region 179 serves to minimize the high pressure and further spreads the change in pressure over a wider region using rounded nose 178. The minimization of this high pressure region reduces fan cutoff noise at cutoff region 179.

Cutoff region 179 in some embodiments includes a rounded nose 178. The rounded nose 178 aids in separating airflow 109 from fan unit 180 without creating significant turbulence. The rounded aspect of the nose 178 serves to divide or deflect airflow 109.

As shown in FIG. 4, fan unit 180 can comprise motor 184 and an impeller 186 designed to create the airflow. It should be understood that motor 184 and impeller 186 can be chosen according to any manner of design and operational requirements. Impeller 186 can be directly driven off a motor shaft 182 (see in FIG. 3). Impeller 186 may be directly adjacent to, or distanced from motor 184 via a drive shaft with length L. Length L of drive shaft 182 can vary depending upon the width of air cleaner 100, the specific impeller used, and by design and operational requirements. It is important to note that the further the impeller is distanced from motor 184, the greater the strain on motor 182 and bearings therein. Impeller 186 can comprise a cross-flow blower 186. Advantageously, the use of a cross-flow blower distributes the strain of the rotors evenly across the drive shaft, thereby reducing the burden on the motor. Cross-flow blower 186 can be supported by a bearing 188. Bearing 188 can be disposed opposite motor 185. Fan unit 180 can be controlled to create and regulate the airflow. Fan unit 180 can include variable speed settings, including low, medium and fast speeds. Fan unit 180 can be affixed to any manner of mount (not shown) in air channel 107. Fan unit 180 can be removably or permanently affixed to the mount.

FIG. 4 also illustrates a UV LED assembly 150 that can include a UV frame 152. One or more circuit boards 154 can be electrically connected to a power source (not shown) and fixed to UV frame 152. Circuit board 154 can provide one or more UV LEDs 156 to provide UV illumination. UV illumination from UV LED assembly 150 can contained wholly within housing 106. In some embodiments, the UV illumination may be visible to a user, for example, through air inlet 110 during normal operation of air cleaner 100. In a preferred embodiment, when the UV illumination is visible outside the housing, only UV-A illumination is produced by the UV light source.

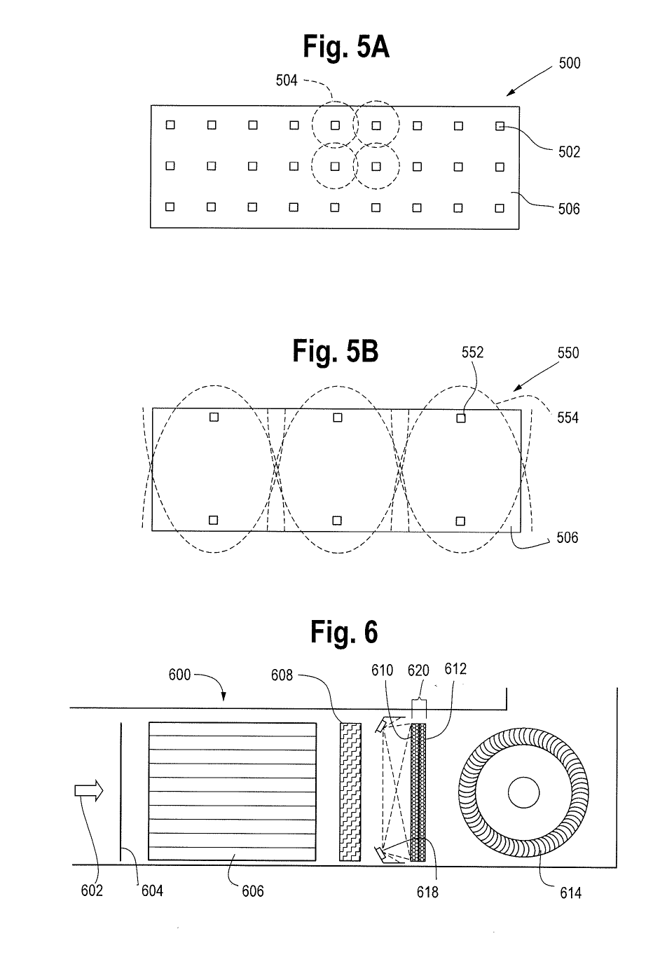

FIG. 5A is one embodiment of a UV LED assembly 500 that provides a high-density distribution of UV LEDs 502. In some embodiments, UV LEDs 502 can comprise low intensity UV LEDs. A high-density distribution can increase the intensity of the illumination provided by UV LEDs 502. In some embodiments, UV LEDs 502 can provide light in the UV-A spectrum.

FIG. 5B is one embodiment of a UV LED assembly 550 that provides a sparse or low-density distribution of UV LEDs 552. In some embodiments, UV LEDs 552 can comprise high intensity UV LEDs. A sparse distribution can provide a desired intensity of UV illumination without using a large number of UV LEDs 552. In some embodiments, UV LEDs 552 can provide light in the UV-A spectrum.

FIG. 6 illustrates another embodiment of an air cleaner. Air cleaner 600 includes an air channel 602. A pre-filter 602, an electrostatic precipitator 604, a VOC filter 608, a post-filter 620 comprising a photocatalytic oxidation (PCO) element 610 and a ozone decomposing element 612, and an air moving unit 614 can be disposed in the air channel. A UV LED assembly 616 can radiate UV light on PCO element 610 using a UV LED 618. UV LED 618 can comprise a plurality of UV LEDs. One or more of UV LED assembly 616 can be disposed in air cleaner 600. The quantity of UV LED 418 and/or UV LED assembly 616 can be optimized to provide the correct intensity of illumination to PCO element 610. In some embodiments, UV LED 618 can provide light in the UV-A spectrum.

The UV illumination can be supplied by UV LED assembly 150, and may be configured to irradiate, sanitize, or otherwise disinfect a variety of infestation agents that may be present within airflow. These agents are capable of passing through pre-filter 120, electrostatic precipitator 130, and post-filter 140, or alternatively generate ozone. In general, UV light wavelengths are considered to have a wavelength that is less than about 400 nm. UV light is considered beyond the range of visible light. The UV light waves can have wavelengths of 400-320 nm, 320-280 nm, or 280-100 nm, and are normally referred to as UV-A, UV-B, and UV-C waves respectively. Preferably, the UV light waves are UV-A with wavelengths of 400-320 nm. The dosage of UV light (in terms of millijoules per square centimeter or “mJ/cm”) is a product of light intensity (or irradiance) and exposure time. Intensity is measured in microwatts per square centimeter (μW/cm²), and time is measured in seconds. The light source may be, for example, a generally U-shaped, 35-watt, high-output, no-ozone bulb (not shown) suitable for radiating light in the selected UV wavelength range of light, or a series of LED UV lights as seen in FIG. 5A, FIG. 5B and FIG. 6. In some embodiments, a single linear bulb or multiple linear or shaped bulbs can be employed. If UV LEDs are used, the LEDs may comprise 1, 2, 3, 4, 5, 6 or more UV LEDs. The lights may be configured in series or in parallel. The loss of power to one bulb may or may not be sufficient to shut down the remaining bulbs. LEDs adapted to generate light waves in the UV-A range are available from Advanced Optoelectrontics Technology, Taiwan, R.O.C. as part number AOT-CN5050UV27D-Z0.

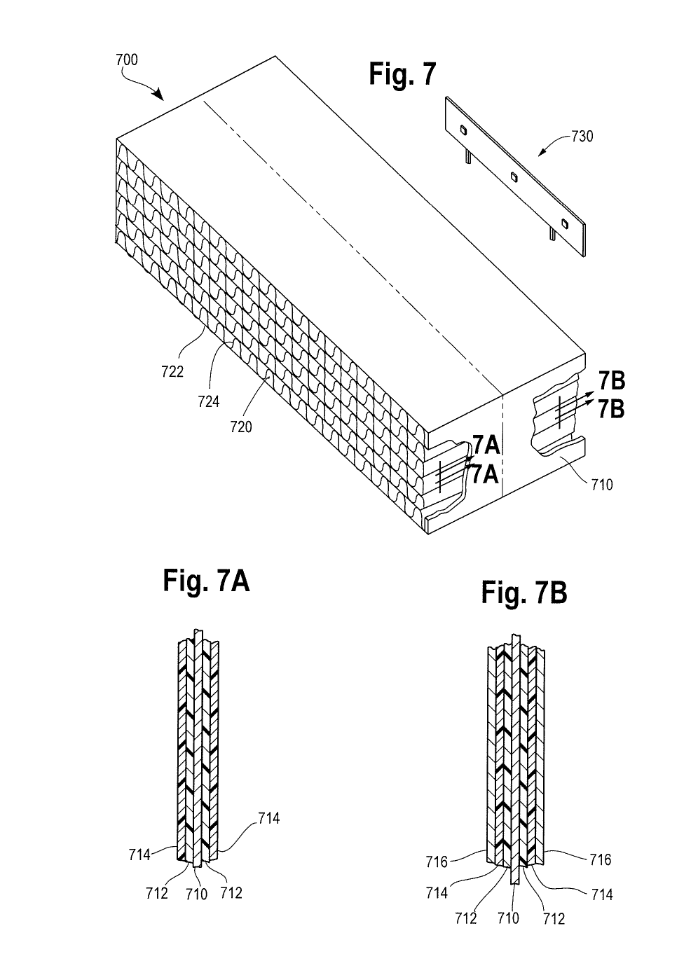

FIG. 7, FIG. 7A and FIG. 7A show a post-filter 700 according to an embodiment. Post-filter 700 can comprise any manner of desired filter element. In one embodiment, post-filter 700 comprises a substrate 710. Substrate 710 can comprise a fiber, a mesh, a woven filter, paper, cloth, porous material, or porous structure, for example. Post-filter 700 can comprise a HEPA filter, an allergen air filter, an electrostatic air filter, a charcoal filter, or an anti-microbial filter, as previously described. As before, post-filter 700 can be treated with a germicide, fungicide, bactericide, insecticide, etc. Post-filter 700 can have length L, height H, and width W. Post-filter 700 can be capable of filtering impurities or particulates with an average diameter of at least 0.1, 0.3, 0.5, 1.0, 5.0, 10.0, 100 microns or greater, including impurities or particulates with an average diameter of 0.001, 0.01, 0.1, 1.0 millimeters or greater.

Post-filter 700 can include one or more of an odor filtration, VOC and/or ozone filtration element. Post-filter 700 can use a catalyzing compound for generating and removing ozone. Post-filter 700 can use a catalyzing compound for removing VOCs. Post-filter 700 includes air passages 720 which filter odors, VOCs or ozone. Air passages 720 are formed by series of substantially serpentine sheets 724 interspersed with substantially planar divider sheets 722. Sheets 722 and 724 can comprise any suitable materials. Substrate 710 can comprise any number of serpentine sheets 724 and planar divider sheets 722, wherein the substrate 722 can be formed to a desired shape and size. In some embodiments, air passages 720 can include any cross-sectional shape, including octagonal, hexagonal, circular, irregular, etc. In one embodiment, substrate 710 is formed of a metal matrix, such as an aluminum matrix, for example. The aluminum matrix allows some compression, wherein the aluminum matrix can accommodate some shaping. In another embodiment, the substrate 710 is formed of a ceramic/paper matrix. The ceramic/paper matrix advantageously can be impregnated with a higher concentration of removal components than a metal matrix.

As illustrated in FIG. 7B, post-filter 700 can comprise a substrate 710 (such as a three-dimensional matrix, for example) that includes photocatalytic oxidation (PCO) layer 716 deposited on substrate 710. The PCO layer 716 is activated by UV light supplied by, for example, a UV LED assembly. PCO layer 716 may react with water vapor from the air to release peroxide. Photocatalytic oxidation utilizes ultraviolet or near-ultraviolet radiation to promote electrons from the valence band into the conduction band of a metal oxide semiconductor. Decomposition of VOCs takes place through reactions with molecular oxygen or through reactions with hydroxyl radicals and super-oxide ions formed after the initial production of highly reactive electron and hole pairs. Thus PCO layer 716 extends the life of post-filter 700. For example, post-filter 700 can comprise an ozone catalyst layer 714 deposited on substrate 710. In this embodiment, post-filter 700 can remove a significant amount of the ozone in an airflow. Post-filter 700 can also include a VOC decomposition layer deposited on substrate 712. As a result, post-filter 700 removes VOCs in an airflow by a process of catalyzation. Post-filter 700 can further remove odors from the airflow. The odor removal can be by catalyzation or adsorption. Because post-filter 700 substantially removes ozone, VOCs, and odors from an airflow, an air cleaner can remove a very high proportion of contaminants that can cause odors, irritation, or health problems. In addition, VOCs are substantially removed from the air, removing the health risks that they represent. As illustrated in FIG. 7A, a portion of substrate 710 is not covered by PCO layer 716. The portion of substrate 710 that includes PCO layer 716 can be illuminated by a UV LED 730. The illumination from UV LED 730 can catalyze the photo-catalytic oxidation reaction.

The ozone decomposing catalyst layer can be deposited over the entire substrate, or a portion thereof. The ozone decomposing catalyst layer can be deposited over 10, 20, 30, 40, 50, 60, 70, 80, 90, 95 or 100 percent of the entire substrate of post-filter 700. The VOC decomposing catalyst layer can be deposited over the entire substrate, or a portion thereof. The VOC decomposing catalyst layer can be deposited over 10, 20, 30, 40, 50, 60, 70, 80, 90, 95 or 100 percent of the entire substrate of post-filter 140. The PCO catalyst layer can be deposited over a portion of the surface area of the entire substrate. The PCO catalyst layer can be deposited over 10, 20, 30, 40, 50, 60, 70, 80, 90, or 95 percent of the entire substrate of post-filter 700. In an embodiment, the PCO catalyst layer can be deposited over 50 percent of the surface of the substrate. The remaining 50 percent of the surface of the substrate can comprise the VOC decomposing catalyst layer. The catalyst layers can be applied simultaneously or sequentially. The catalyst layers can be applied in any order. In some embodiments, the PCO catalyst is the outside layer for a portion of the surface area of the substrate, for example, 50% of the surface area. In some embodiments, the ozone removal layer can be applied prior to the VOC removal layer that is applied prior to the PCO catalyst layer. In other embodiments, the VOC removal layer can be applied prior to the ozone removal layer that is applied prior to the PCO catalyst layer.

For example, post-filter 700 can include some manner of carbon, zeolite, or potassium permanganate filter or filter component for odor removal. In addition, post-filter 700 can include an odor emitting element. For example, post-filter 700 can include a perfume packet or cartridge portion that emits a desired perfume (or other scent). Therefore, post-filter 700 can comprise one or more of a mechanical filter element, an odor filtration element, and an odor emitting element.

Additionally, in one embodiment, ozone decomposing material 712 includes a metal oxide material deposited on substrate 710. Ozone reacts with the metal oxide and decomposes in a catalytic reaction. In one embodiment, ozone decomposing material 712 comprises manganese oxide (MnO₂). In another embodiment, ozone decomposing material 712 comprises titanium dioxide (TiO₂). However, it should be understood that ozone decomposing material 712 can comprise any manner of suitable metal oxide, such as, but not limited to Al₂O₃, SiO₂, TiO₂, Fe₂O₃, ZnO. In another embodiment, the ozone decomposing catalytic material includes two or more catalytic materials for ozone removal. Furthermore, post-filter 700 can comprise a HONEYCLE material, available from NCl Mfg., Inc., Scottsboro, Ala. Post-filter 700 can comprise a NHC material, available from Nikki-Universal Co., Ltd., Tokyo, Japan. Post-filter 700 can comprise a material available from Dongguan UniClear New-material Co., Ltd., Dongguang, Guangdong P.R.C.

In some embodiments, post-filter 700 can comprise a single VOC removal material. In another embodiment, the VOC catalytic material includes two or more catalytic materials for VOC removal. Post-filter 700 can comprise a MnO₂ material. However, it should be understood that the VOC removal material can comprise any manner of suitable metal oxide, such as, but not limited to Al₂O₃, MnO₂, SiO₂, TiO₂, Fe₂O₃, ZnO. Thus, post-filter 700 may optionally include a single removal element that simultaneously removes ozone, VOCs, and odors from the airflow.

The post-filter receptacle (not shown) can comprise a drop-in or slide-in receptacle, for example, wherein post-filter 700 (or some manner of post-filter system) fits to some manner of rails, grooves, pins, etc., which receive and retain post-filter 700. In the embodiment of air cleaner 100 shown in FIG. 2, post-filter 140 slides into the electrostatic post-filter receptacle (not shown).

Additionally, an air cleaner may contain additional accessories which aid in the function or maintenance of the air cleaner. Non-limiting examples of such accessories include remote controls, cleaning brushes, handles, screw drivers, cords, etc. The air cleaner housing may optionally be configured to further house optional accessories in discrete interior or exterior drawers, compartments or chambers, allowing for immediate access and use of any accessory. The optional accessories may be held in the drawers, compartments or chambers via tie-downs, clamps, cut-outs, etc.

The convertible air cleaner can be implemented according to any of the embodiments in order to obtain several advantages, if desired. The invention can provide an effective and efficient convertible air cleaner. Advantageously, the independent components enable the installation and removal of components. For example, the components can be selected and added in order to obtain a special or custom configuration of the air cleaner. In addition, the airflow will be optimally cleaned before reaching the fan unit, extending motor life and lowering operating costs. Finally, the air cleaner is capable of efficiently and thoroughly cleaning the air whether the air cleaner is oriented in a tower or a non-tower position, thereby reducing redundancy, extending the life of the motor and reducing operation and energy costs.

The various embodiments described above are provided by way of illustration only and should not be constructed to limit the invention. Those skilled in the art will readily recognize the various modifications and changes which may be made to the present invention without strictly following the exemplary embodiments illustrated and described herein, and without departing from the true spirit and scope of the present invention, which is set forth in the following claims.

What is claimed is:

1. An air cleaner comprising:
an air channel in the air cleaner;
an air moving unit configured to create an airflow in the air channel;
a photo-catalytic oxidation (PCO) element disposed in the airflow; and
an Ultraviolet A (UV-A) Light Emitting Diode (LED) to illuminate the PCO element.

2. The air cleaner of claim 1, with the PCO element causing decomposition of water vapor in the airflow.

3. The air cleaner of claim 1, with the PCO element being supported by pellets, mesh, screen, cloth, paper, a porous substrate or a combination thereof.

4. The air cleaner of claim 1, with the PCO element comprising titanium dioxide.

5. The air cleaner of claim 1, with the UV-A LED being disposed outside of the air channel.

6. The air cleaner of claim 1, further comprising a decomposing element disposed in the air flow.

7. The air cleaner of claim 1, further comprising:
a filter; and
a decomposing element,
wherein the PCO element and the decomposing element are supported by the filter.

8. The air cleaner of claim 7, with the filter including first and second opposite faces, the decomposing element being disposed on the first face of the filter and the PCO element being disposed on the second face of the substrate.

9. The air cleaner of claim 8, with the filter further including air passages between the first face and the second face.

10. The air cleaner of claim 9, with the honeycomb ? having a length that is greater than a length of the PCO coating on the honeycomb.

11. The air cleaner of claim 7, with the decomposing element being disposed in the airflow downstream of the PCO element.

12. The air cleaner of claim 7, wherein the decomposing element comprises an ozone decomposition element.

13. The air cleaner of claim 7, wherein the decomposing element comprises a VOC decomposition element.

14. The air cleaner of claim 7, wherein the decomposing element comprises an odor removal element.

15. The air cleaner of claim 7, wherein the decomposing element comprises an ozone decomposition element and a VOC removal element.

16. The air cleaner of claim 1, with the UV-A LED being operable to emit UV light comprising wavelengths from 320 nanometer (nm) to 400 nm.

17. The air cleaner of claim 1, wherein the PCO element comprises titanium dioxide and the UV-A light emitting diode is operable to emit UV light having an intensity sufficient to energize the titanium dioxide to cause photo-catalytic oxidation.

18. The air cleaner of claim 1, further comprising an air filter element adapted to capture particulates having an average diameter less than 1 millimeter from the airflow.

19. The air cleaner of claim 1, further comprising an electrostatic precipitator disposed in the air flow upstream of the PCO element.

20. An air cleaner comprising:
an air channel in the air cleaner;
an air moving unit configured to create an airflow in the air channel;
an VOC decomposing element located in the air channel;
a PCO element comprising titanium dioxide in contact with the VOC decomposing element; and
a UV LED to illuminate the PCO element;
wherein UV LED illumination catalyzes a reaction in the PCO element sufficient to clean the VOC decomposing element.

21. The air cleaner of claim 20, with the UV LED being operable to emit UV light comprising wavelengths from 320 nanometers (nm) to 400 nm.

22. The air cleaner of claim 20, with the UV LED having a mean time between failure (MTBF) of greater than one year or 84600 hours.

23. The air cleaner of claim 20, with the VOC decomposing element having a MTBF of greater than one year or 84600 hours.

24. The air cleaner of claim 20, with the PCO element having a MTBF of greater than one year or 84600 hours.

25. The air cleaner of claim 20, with the PCO element spanning the air channel.

26. A substrate for being disposed in an air cleaner, the substrate comprising:
a first coating comprising a VOC decomposing catalyst;
a second coating comprising an ozone decomposing catalyst; and
a third coating comprising titanium dioxide.

27. The substrate of claim 26, with the substrate comprising two opposite faces and a plurality of air channels extending between the two opposite faces.

28. The substrate of claim 27, with a length of one of the air channels is greater than a length of the third coating.

29. The substrate of claim 26, further comprising a frame disposed around the substrate.

30. The substrate of claim 26, comprising a plurality of serpentine metal sheets being joined together with a plurality of gaps between adjacent metal sheets to form the substrate.
